(19) United States
(12) Patent Application Publication
(10) Pub. No.: US 2005/0124938 A1
(43) Pub. Date: Jun. 9, 2005

(54) One-shot needle safety cover

(76) Inventor: Chang-Ming Yang, Junan Jen (TW)

Correspondence Address: HDSL, 4331 STEVENS BATTLE LANE, FAIRFAX, VA, 22033, US

(21) Appl. No.: 10/989,881

(22) Filed: Nov. 16, 2004

(30) Foreign Application Priority Data
Nov. 17, 2003 (CN) 200320105275.9

(51) Int. Cl. A61M 5/32

(52) U.S. Cl. 604/192

(57) ABSTRACT

A one-shot needle safety cover using a rotational or a drawing method to enclose a used needle. The safety cover can be applied to a syringe, butterfly needle, blood collector, intravenous catheter, and arteriovenous fistula. In the rotating method, by means of a rotating axle a needle can be enclosed inside or exposed from a needle cap. In the drawing method, by means of an orienting member a butterfly needle can be drawn back and forth along a chamber of a needle cover. The present invention is easy to operate, more safe, and is able to prevent a needle from being used a second time.

### BACKGROUND OF THE INVENTION

The present invention relates in general to a needle cover, and more particularly, to a one-shot needle safety cover that is easy to operate, more safe, and is able to prevent a needle from being used a second time.

A conventional syringe, whether it is integrally formed with a needle or comprises a separate needle, utilizes a needle cap to protect the needle and to prevent medical personal from injury. The needle cap must be removed before proceeding with a blood drawing or an injection. After a blood drawing or an injection, the needle must be covered by the needle cap before disposal in order to prevent the danger of an exposed needle. The needle cap must be fully removed from the syringe during a treatment. However, the needle cap is easily lost. Further, placing the needle cap over the needle is dangerous. A medical person could be pierced inadvertently, thereby becoming infected. In addition, the conventional syringe is easily used for a second time.

### BRIEF SUMMARY OF THE INVENTION

The present invention provides a one-shot needle safety cover that is easy to operate, more safe, and is able to prevent the needle from being use a second time.

The one-shot needle safety cover of the present invention includes a rotating member and a fixing member. The rotating member has a needle cap and a rotating arm extending from one side of the needle cap and a rotating axle connecting to the body of the rotating arm. The needle cap has a receptive space formed therein and at least one slot cut therefrom. The slot is parallel with the rotating arm. The rotating axle has an enlarged head at one end thereof.

The fixing member has an axle hole and a fixing hole formed at one side thereof and a protruding sleeve portion formed at the other side thereof. The axle hole and the fixing hole are connected by a passage channel. A retaining portion is formed at the bottom of the passage channel adjacent to the fixing hole. The sleeve portion has a sleeve hole formed in the middle thereof and a receptive opening formed at one end thereof adjacent to the passage channel.

The rotating axle is inserted into the axle hole of the fixing member, with the enlarged head retained by the wall surface of the fixing member, and the sleeve hole of the sleeve portion is sleeved over either the top of a syringe or the bottom of a needle.

The objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These as well as other features of the present invention will become more apparent upon reference to the drawings therein:

FIG. 1 is an exploded view of a one-shot needle safety cover in accordance with the present invention.

FIG. 2 is a perspective view of the one-shot needle safety cover.

FIG. 3 is a side view of a syringe with the one-shot needle safety cover installed.

FIG. 4 is a perspective view of the syringe and the one-shot needle safety cover of FIG. 3.

FIG. 5 is a perspective view of the one-shot needle safety cover, illustrating a manipulation of the safety cover.

FIG. 6 is a perspective view of the one-shot needle safety cover, illustrating another manipulation of the safety cover.

FIG. 7 is an exploded view of the first embodiment of the one-shot needle safety cover.

FIG. 8 is a perspective view of the second embodiment of the one-shot needle safety cover.

FIG. 9 is a perspective view of the third embodiment of the one-shot needle safety cover, illustrating a manipulation thereof.

FIG. 10 is a perspective view of the fourth embodiment of the one-shot needle safety cover.

FIG. 11 is a perspective view, partially broken away, of another one-shot needle safety cover in accordance with the present invention.

FIG. 12 is an exploded view of the one-shot needle safety cover shown in FIG. 11.

FIG. 13 is cross-sectional view of the one-shot needle safety cover shown in FIG. 11.

FIG. 14 is a perspective view of the one-shot needle safety cover shown in FIG. 11, illustrating a manipulation thereof.

FIG. 15 is a perspective view of yet another one-shot needle safety cover in accordance with the present invention.

FIG. 16 is an exploded view of the one-shot needle safety cover shown in FIG. 15.

FIG. 17 is a perspective view of the one-shot needle safety cover shown in FIG. 15, illustrating a manipulation thereof.

FIG. 18 is a perspective view of the one-shot needle safety cover shown in FIG. 15, illustrating another manipulation thereof.

FIG. 19 is an exploded view of the first embodiment of the yet another one-shot needle safety cover.

FIG. 20 is an exploded view of the second embodiment of the yet another one-shot needle safety cover.

FIG. 21 is an exploded view of the third embodiment of the yet another one-shot needle safety cover.

### DETAILED DESCRIPTION OF THE INVENTION

Reference will now be made in detail to the preferred embodiments of the present invention, examples of which are illustrated in the accompanying drawings. Wherever possible, the same reference numbers are used in the drawings and the description to refer to the same or like parts.

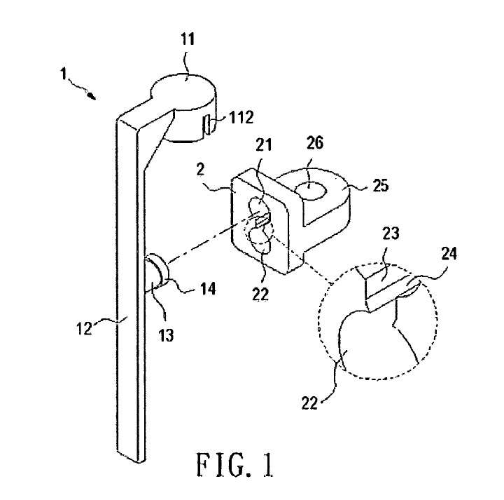

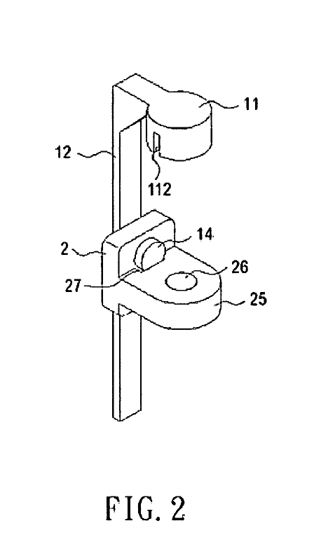

Referring to FIGS. 1 through 3, a one-shot needle safety cover in accordance with the present invention can be applied to a medical device with a needle 3 that includes, but is not limited to a syringe, butterfly needle, blood collector, intravenous catheter, and arteriovenous fistula. The safety cover comprises a rotating member 1 and a fixing member 2.

The rotating member 1 comprises a needle cap 11, a rotating arm 12 extending from one side of the needle cap 11, and a rotating axle 13 connecting to the body of the rotating arm 12. The needle cap 11 has a receptive space 111 formed therein, and at least one slot 112 cut therefrom. The slot 112 is parallel with the rotating arm 12. The rotating axle 13 has an enlarged head 14 at one end thereof for pivotally connecting the rotating member 1 to the fixing member 2.

The fixing member 2 has an axle hole 21 and a fixing hole 22, which are connected by a passage channel 23. A retaining portion 24 is formed about the bottom of the passage channel 23 adjacent to the fixing hole 22. Further, the fixing member 2 has a protruding sleeve portion 25 formed at one side thereof. The sleeve portion 25 has a sleeve hole 26 formed in the middle thereof and a receptive opening 27 formed at one end thereof adjacent to the passage channel.

To assemble the preferred embodiment, the rotating axle 13 is inserted into the axle hole 21 of the fixing member 2, with the enlarged head 14 retained by the wall surface of the fixing member 2. The rotating member 1 is thereby pivotally connected to the fixing member 2. The sleeve hole 26 of the sleeve portion 25 is sleeved over the top of the medical device. Alternatively, the sleeve portion 25 is integrally formed with the medical device, instead connected to that by means of the sleeve hole 26.

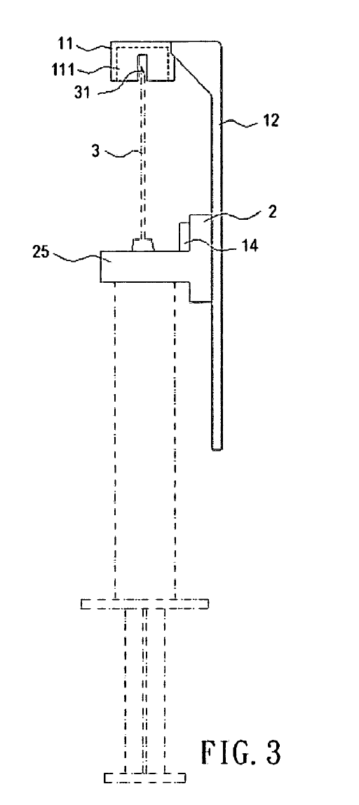

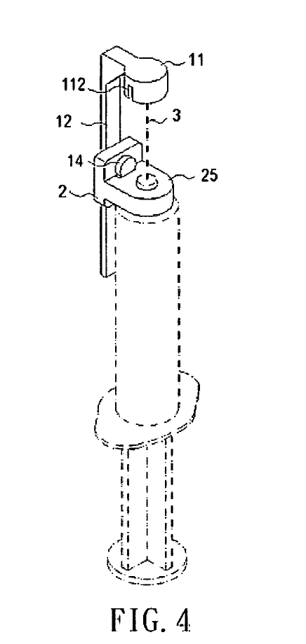

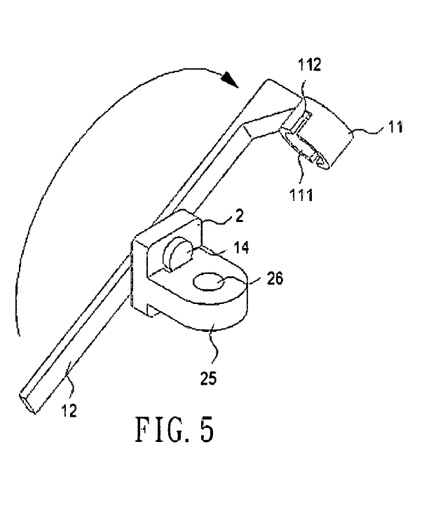

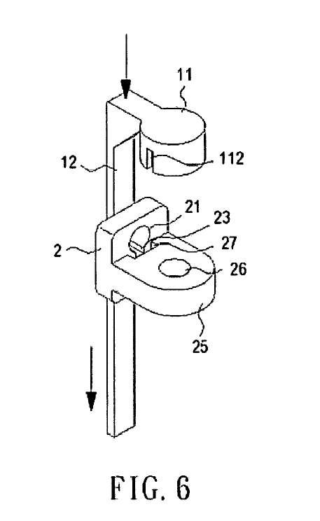

Referring to FIGS. 3 through 6, the usage of the preferred embodiment is illustrated. Initially, the safety cover is assembled onto a syringe by inserting the top of the syringe into the sleeve hole 26 of the sleeve portion 25. The syringe is then rotated around the sleeve hole 26 until an oblique plane 31 of the tip of the needle 3 is opposite the rotating arm 12, as shown in FIG. 3. Such that, the oblique plane 31 of the needle 3 will remain facing outward while the needle 3 is inserted into a patient's body. The oblique plane 31 is kept opposite to the rotating arm 12 so that the rotating arm 12 is oriented above the syringe during an injection. The injection, therefore, will not be hindered by the rotating arm 12. Further, because the tip of the needle 3 is stored inside the rotating member, the rotating member 1 is then rotated around the rotating axle 13 in order to expose the tip of the needle 3 through the slot 112. After these procedures, the syringe can proceed with either a drug injection or a blood withdrawing. After utilization, the rotating member 1 is rotated around the rotating axle 3 back to its former position. The rotating member 1 is further pressed down, and the rotating axle 13 is thereby pushed into the fixing hole 22 through the passage channel 23. The retaining portion 24 retains the rotating axle 13 and prevents it from being outward. The tip of the needle 3 is thereby stored inside the receptive space 111 of the rotating member 1. The tip of the needle 3 cannot be exposed from the rotating member 1 even if a torque is applied to the rotating member 1, because the tip of the needle 3 is blocked by the wall above the slot 112. Accordingly, the syringe prevents the danger of a second use. In addition, a small amount of the patient's blood or the drug delivered is likely to remain in the needle 3. Consequently, a water absorbing element is disposed inside the receptive space 111. The water absorbing element can absorb the remained blood or drug, and prevent dangerous incidents.

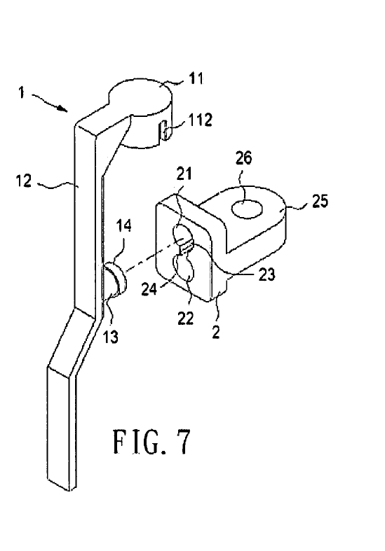

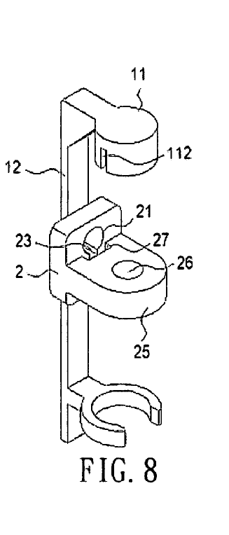

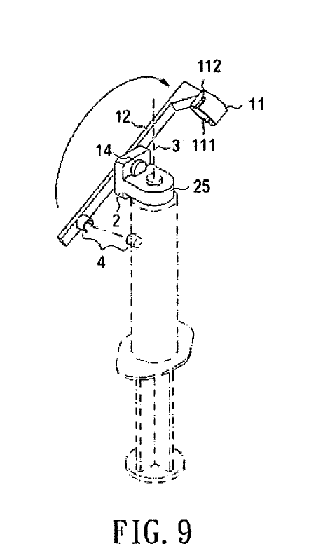

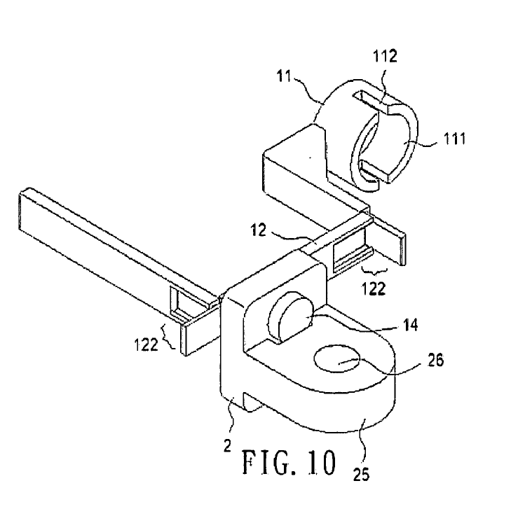

Referring to FIGS. 7 through 10, different preferred embodiments are shown. In FIG. 7, the bottom of the rotating arm 12 extends outwardly. In FIG. 8, at the bottom of the rotating arm, the rotating member 1 can further comprise a clip portion 121 that is able to clasp the outer wall of the syringe. In FIG. 9, the one-shot needle safety cover further comprises a securing means 4 that is disposed on both the rotating arm 12 and the syringe for fixing the rotating member 1 to the syringe. There are many methods to connect the securing means 4, which include, but are not limited to, snap-on, sleeve-on, sleeve connecting, engagement, joint, junction, and retaining method. Connecting the rotating arm 12 to the syringe prevents the rotating member 1 from rotating before use. The danger caused by inadvertent exposure of the needle 3 is, therefore, eliminated. Furthermore, as shown in FIG. 10, the rotating member 1 further comprises at least one fold portion 122. The length of the rotating arm 12 can be shortened by means of the fold portion 122 in order to facilitate the blood drawing or the injection.

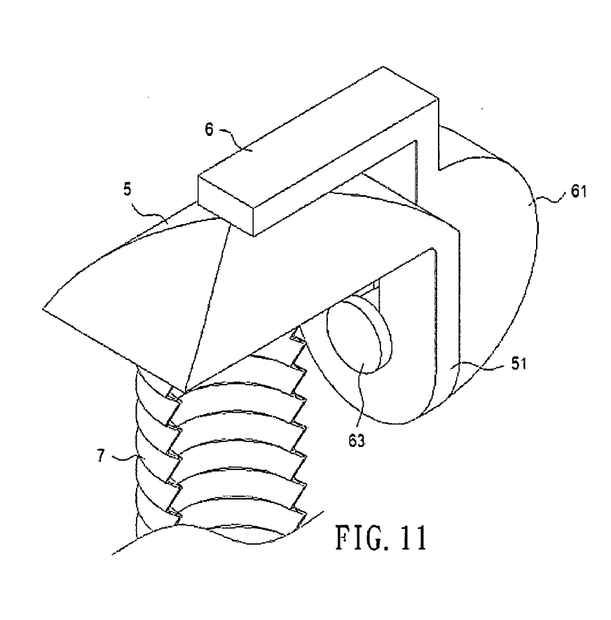

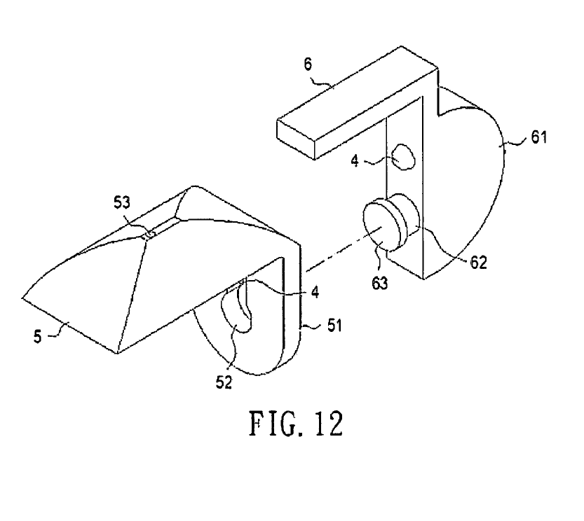

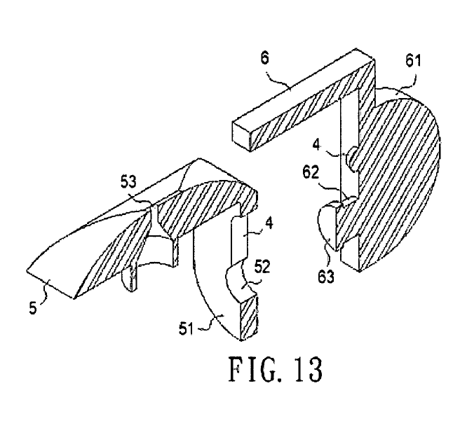

Referring to FIGS. 11 through 13, another one-shot needle safety cover of the present invention comprises a protective cap 5, a rotating element 6, and an extending sleeve 7.

The protective cap 5 has a through hole 53 bored therefrom. A vertical portion 51 extends from one side of the protective cap 5. The vertical portion 51 has an axle hole 52 formed in the middle thereof.

The rotating element 6 has an extending portion 61 formed at one side thereof. A rotating axle 62 extends from the extending portion 61, with an enlarged head 63.

By means of an extending sleeve 7, the protective cap 5 can be connected to the medical device. The rotating axle 62 is installed into the axle hole 52, thereby pivotally connecting the rotating element 6 to the protective cap 5. The enlarged head 63 is retained by the inner wall surface of the vertical portion 51. The rotating element 6 is oriented over the through hole 53 of the protective cap 5. In addition, the one-shot needle safety cover further comprises a securing means 4 that is disposed on the rotating element 6 and the protective cap 5 for fixing them together. There are many methods to connect the securing means 4, which include, but are not limited to, snap-on, sleeve-on, sleeve connecting, engagement, joint, junction, and retaining method.

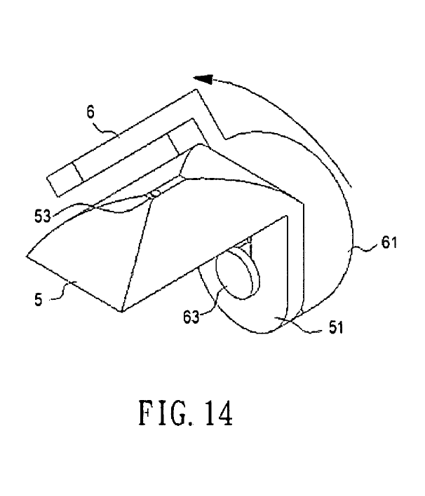

Referring to FIG. 14, the usage of the one-shot needle safety cover is illustrated. The extending sleeve 7 is sleeved onto a syringe with a needle 3, with the protective cap 5 and the rotating element 6 disposed on the top of the needle 3. The tip of the needle 3 is received by the through hole 53 of the protective cap 5. To use the syringe, the securing means 4 is disconnected, the rotating element 6 is rotated around the rotating axle 62, and the through hole 53 of the protective cap 5 is exposed. Further, the protective cap 5 and the extending sleeve 7 are pulled down, and thereby the needle 3 protrudes from the through hole 53. The syringe is, therefore, able to proceed with a blood drawing or an injection. After use, the protective cap 5 and the extending sleeve 7 are returned to their former positions, and the rotating element 6 is further rotated back to cover the through hole 53. The danger of an exposed needle is prevented. Additionally, the extending sleeve 7 may include a circular fastener at the bottom thereof for connecting to the syringe.

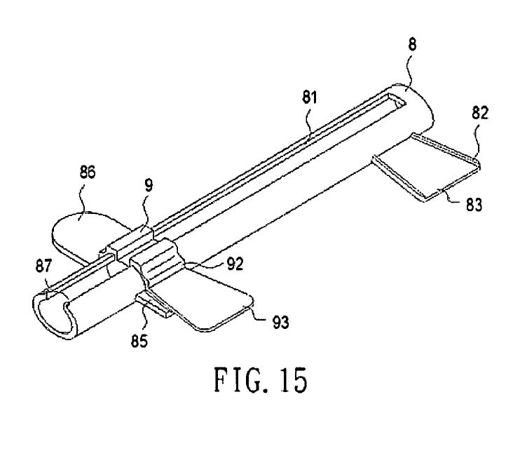

Referring to FIGS. 15 through 18, yet another one-shot needle safety cover of the present invention is applied to the butterfly needle. The one-shot needle safety cover comprises a needle cover 8 and an orienting member 9.

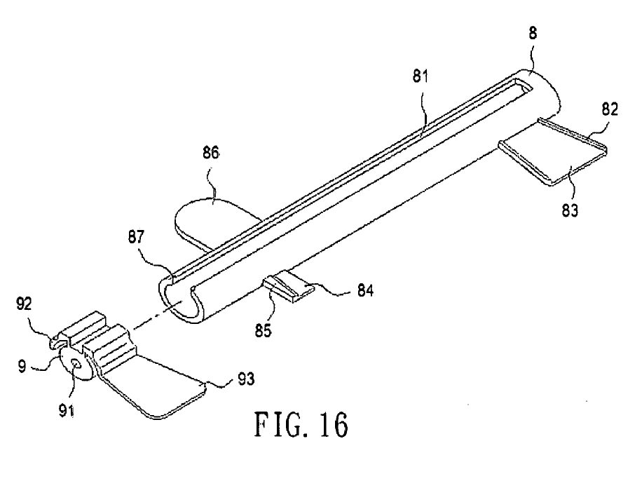

The needle cover 8 has a chamber 81 formed therein. On one side of the needle cover 8, a fixing plate 82, having a concave space 83, connects to one end of the needle cover 8. On the same side of the needle cover 8, a retaining plate 84, having a retaining block 85, connects to the other end of the needle cover 8. On the other side of the needle cover 8 opposite the retaining plate 84, a handle portion 86 connects to the needle cover 8. In addition, at the inlet of the chamber 81, a retaining portion 87 is formed, as shown in FIG. 16.

The orienting member 9 comprises a sleeve hole 91 bored therefrom, and two clasps 92 which extend from both sides of the top thereof, respectively. One clasp 92, opposite to the fixing plate 82, has a clip tab 93 extending therefrom.

The orienting member 9 uses the sleeve hole 91 to hold the butterfly needle. This combination can be further inserted into the chamber 81 and moved along the chamber 81 until the clip tab 93 exceeds totally the retaining plate 84. The clasp 92 vices the wall of the needle cover 8. The retaining block 85 retains the clip tab 93. The needle 3 of the butterfly needle is thereby received inside the needle cover 8. Alternatively, the orienting member 9 can be integrally formed with the butterfly needle.

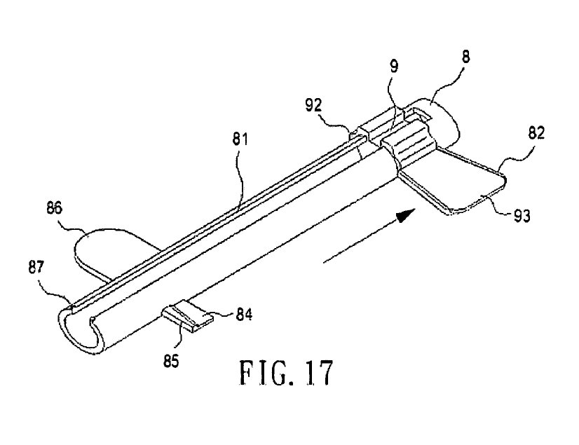

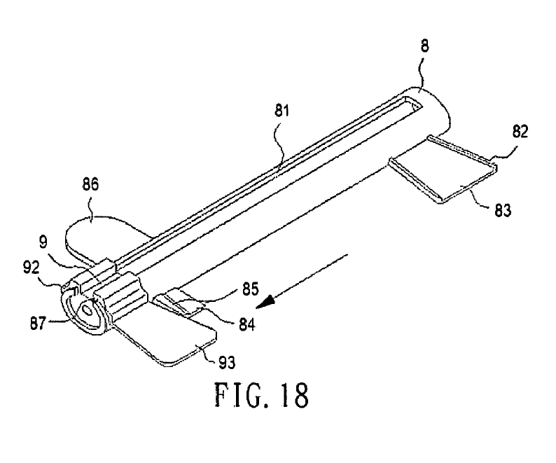

To use the butterfly needle, the clip tab 93 is pulled forward thereby dragging both the orienting member 9 and the butterfly needle to the front of the needle cover 8, as shown in FIG. 17. The needle 3 of the butterfly needle, therefore, protrudes from the needle cover 8. Further, the clip tab 93 is snapped into the concave space 83 of the fixing plate 82 in order to fix the orienting member 9 to the needle cover 8. Therefore, the orienting member 9 will not slip during a blood drawing or an injection. After use, the clip tab 93 is disconnected from the fixing plate 82, and further pulled backward until the front edge thereof exceeds the rear of the retaining plate 84. Consequently, the clip tab 93 will be blocked by the retaining block 85, as shown in FIG. 18. Because the orienting member 9 cannot move forward, the danger of a protruding needle 3 is prevented.

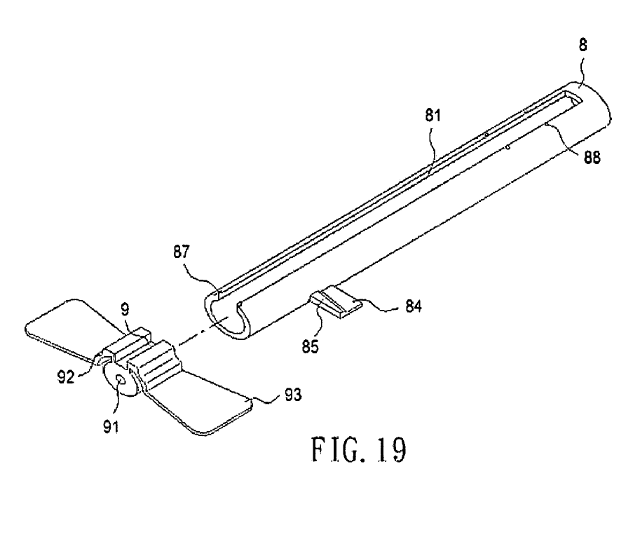

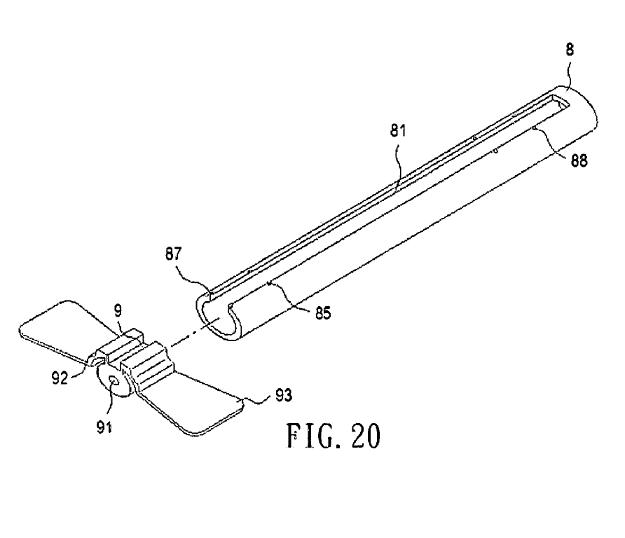

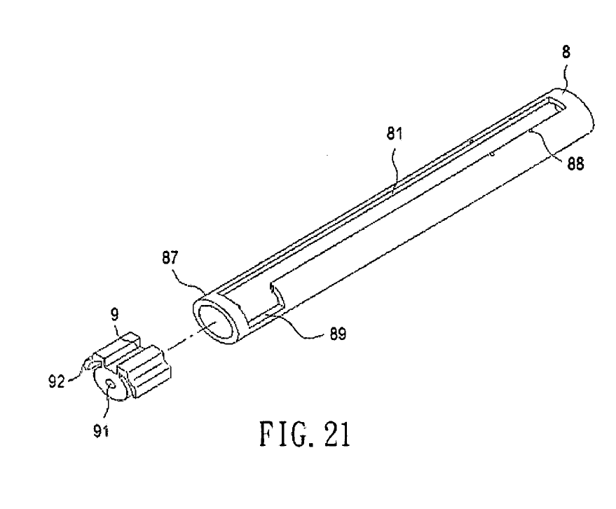

Referring to FIGS. 19 and 21, another three embodiments are shown. In FIG. 19, the handle portion 86 is omitted from the needle cover 8, and another clip tab 93 is added to the orienting member 9. Both clasps 92 have a clip tab 93 extending therefrom. Further, a plurality of orienting points 88 is disposed on the outer edge of the chamber 81 to substitute for the fixing plate 82. In FIG. 20, the retaining plate 84 is replaced with orienting points 85 formed on the outer edge of the chamber 81. In FIG. 21, the clip tabs 93 are omitted from the orienting member 9, and the orienting point 85 is replaced by a trench 89.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

1. A one-shot needle safety cover, being applied to a medical device with a needle, comprising:
a rotating member having a needle cap and a rotating arm extending from one side of the needle cap and a rotating axle connecting to the body of the rotating arm, the needle cap having a receptive space formed therein and at least one slot cut therefrom, the slot parallel with the rotating arm, the rotating axle having an enlarged head at one end thereof; and
a fixing member having an axle hole and a fixing hole formed at one side thereof and a protruding sleeve portion formed at the other side thereof, the axle hole and the fixing hole connected by a passage channel, a retaining portion formed at the bottom of the passage channel adjacent to the fixing hole, the sleeve portion having a sleeve hole formed in the middle thereof and a receptive opening formed at one end thereof adjacent to the passage channel;
wherein the rotating axle is inserted into the axle hole of the fixing member, with the enlarged head retained by the wall surface of the fixing member, and the sleeve hole of the sleeve portion is sleeved over the top of the medical device.

2. The one-shot needle safety cover of claim 1, wherein the medical device is a syringe or blood collector or intravenous catheter or arteriovenous fistula.

3. The one-shot needle safety cover of claim 1, wherein the sleeve portion is integrally formed with the medical device.

4. The one-shot needle safety cover of claim 1, wherein the needle cap has a water absorbing element disposed inside the receptive space thereof.

5. The one-shot needle safety cover of claim 1, wherein the bottom of the rotating arm extends outwardly.

6. The one-shot needle safety cover of claim 1, wherein the rotating member has a clip portion formed at the bottom of the rotating arm to clasp the outer wall of the syringe.

7. The one-shot needle safety cover of claim 1, wherein the rotating member further comprises at least one fold portion.

8. The one-shot needle safety cover of claim 1 including a securing means disposed on the rotating arm and the syringe for fixing the rotating member to the syringe.

9. A one-shot needle safety cover, being applied to a medical device with a needle, comprising:
a protective cap having a through hole bored therefrom and a vertical portion extending from one side thereof, the vertical portion having an axle hole formed in the middle thereof;
a rotating element having an extending portion formed at one side thereof and a rotating axle extending from the extending portion, the rotating axle having an enlarged head; and
an extending sleeve that is sleeved over the medical device, one end thereof connecting to the protective cap, the other end thereof connecting to the medical device;
wherein the rotating axle of the rotating element is inserted into the axle hole of the protective cap, with the enlarged head retained by the wall surface of the vertical portion of the protective cap.

10. The one-shot needle safety cover of claim 9, wherein the medical device is a syringe or blood collector or intravenous catheter or arteriovenous fistula.

11. The one-shot needle safety cover of claim 9 including a securing means disposed on the rotating element and the protective cap for fixing the rotating element to the protective cap.

12. The one-shot needle safety cover of claim 9, wherein the extending sleeve has a circular fastener at the bottom thereof for connecting to the medical device.

13. A one-shot needle safety cover applied to a butterfly needle, comprising:
a needle cover having a chamber therein and a fixing plate at one front side thereof and a retaining plate at one rear side thereof and a handle portion at the other side thereof and a retaining portion at the inlet of the chamber, the fixing plate having a concave space therein, the retaining plate having a retaining block thereon; and
an orienting member having a sleeve hole bored therefrom and two clasps respectively extending from both sides of the top thereof, the clasp corresponding to the retaining plate having a clip tab extending therefrom;
wherein the orienting member uses the sleeve hole to hold the butterfly needle, the combination is inserted inside the chamber with the clasp vicing the wall of the needle cover, and the clip tab is retained by the retaining block.

14. The one-shot needle safety cover of claim 13, wherein the handle portion is omitted from the needle cover, and another clip tab is added to the clasp without the clip tab.

15. The one-shot needle safety cover of claim 13, wherein the fixing plate is replaced with a plurality of orienting points formed on the outer edge of the chamber.

16. The one-shot needle safety cover of claim 13, wherein the retaining plate is replaced with orienting points formed on the outer edge of the chamber.

17. The one-shot needle safety cover of claim 13, wherein the clip tabs are omitted from the orienting member, and the retaining plate is replaced with a trench.

18. The one-shot needle safety cover of claim 13, wherein the orienting member and the butterfly needle are integrally formed.
